(19) United States
(12) Patent Application Publication
(10) Pub. No.: US 2025/0325472 A1
(43) Pub. Date: Oct. 23, 2025

(54) NOVEL COMPOSITIONS

(71) Applicant: DSM IP Assets B.V., Maastricht (NL)

(72) Inventor: Christine MENDROK-EDINGER, Kaiseraugst (CH)

(21) Appl. No.: 19/122,080

(22) PCT Filed: Oct. 27, 2023
(86) PCT No.: PCT/EP2023/080045

§ 371 Date: Apr. 17, 2025

(30) Foreign Application Priority Data
Oct. 28, 2022 (EP) 22204300.2

(51) Int. Cl. A61K 8/98; A61K 8/06; A61K 8/55; A61Q 17/04

(52) U.S. Cl. CPC A61K 8/986; A61K 8/06; A61K 8/553; A61Q 17/04

(57) ABSTRACT

The present invention relates to emulsions containing at least one sunscreen active agent and having improved sun protection factor (SPF) properties, as well as methods of improving sun protection factor (SPF) properties.

The present invention relates to emulsions containing at least one sunscreen active agent and having improved sun protection factor (SPF) properties, as well as methods of improving sun protection factor (SPF) properties.

The use of compositions containing sunscreen active agents (also called sunscreens) is very common nowadays. The sunscreen active agents in such compositions protect keratinous material such as in particular the skin from the harm caused by UV radiation, including harm from both UVA and UVB rays, which are known to not only contribute to skin wrinkling but also to foster the development of skin diseases, such as lupus erythematosus and melanoma and non-melanoma skin cancer. Thus, to get ultimate skin protection, there's a constantly increasing need for sunscreens exhibiting high SPF's (Sun Protection Factor).

A standard measure for determining the amount of protection a composition containing sunscreen active agent provides against UV radiation is the sun protection factor (SPF). However, given the finite amount of approved sunscreen active agents worldwide, increasing SPF of compositions containing sunscreen active agents can be difficult.

Thus, there is an ongoing need for preferably natural ingredients which are able to increase the SPF of sunscreen active ingredients in compositions.

Surprisingly it has now been found that human milk oligosaccharides are able to significantly increase the SPF of an emulsions comprising a phosphate ester emulsifier and at least one sunscreen active ingredient.

Thus, in a first embodiment, the present invention relates to an emulsion composition for keratinous materials (for example, hair or skin) with a phosphate ester emulsifier, which emulsion composition comprises at least one sunscreen active agent and at least one human milk oligosaccharide. Preferably, the composition is in the form of an oil-in-water (O/W) emulsion.

The present invention also relates to methods of increasing the sun protection factor (SPF) properties of an emulsion composition with a phosphate ester emulsifier comprising at least one sunscreen active agent, said method comprising adding at least one human milk oligosaccharide to the composition in an amount effective to increase the SPF properties of the composition.

The invention also relates to a use of a human milk oligosaccharide for increasing the sun protection factor (SPF) of an emulsion composition with a phosphate ester emulsifier comprising at least one sunscreen active agent.

The present invention also relates to methods of preparing emulsion compositions with a phosphate ester emulsifier having improved sun protection factor (SPF) properties comprising adding at least one sunscreen active agent and at least one human milk oligosaccharide to the composition during preparation of the emulsion composition. It is well understood that said improved properties are to be compared to a composition not comprising the human milk oligosaccharide with all the definitions and preferences as given herein.

The present invention further relates to methods of making an emulsion composition with a phosphate ester emulsifier, said method further comprising combining at least one sunscreen active agent and at least one human milk oligosaccharide in said composition.

In accordance with the invention is also the use of the emulsion composition of the invention for protection of skin from the effects skin aging (especially for protection from UV-induced skin aging) such as in particular wrinkles and as a sun protection composition.

Finally, a subject-matter of the invention is a method for the cosmetic treatment of keratinous substances such as in particular the skin, wherein an emulsion composition as defined herein is applied to the said keratinous substances such as in particular to the skin. The method is in particular suitable to protect the skin against the adverse effects of UV-radiation such as in particular sun-burn and/or photoaging.

The term ‘emulsion composition with a phosphate ester emulsifier’, refers to emulsions using a phosphate ester emulsifier as emulsifier or co-emulsifier to stabilize the emulsion composition. The preparation of such emulsions is well known to a person skilled in the art and outlined in the examples.

The term ‘keratinous materials’ means the skin (body, face, contour of the eyes, scalp), head hair, eyelashes, eyebrows, bodily hairs, nails and/or lips. Preferably, in all embodiments the keratinous material is the skin.

The term ‘human milk oligosaccharides’ (HMOs) refers to a family of structurally diverse unconjugated glycans that are highly abundant in and unique to human milk.

Originally, HMOs were proposed to be prebiotic “bifidus factors,” or human milk glycans found to promote growth in Bifidobacterial species of the gut and found uniquely in the stool of breast-fed infants compared to formula fed infants.

The SPF according to the present invention is understood to be measured by the in vitro SPF method as outlined in ISO 24443 (Determination of sunscreen UVA photoprotection in vitro, 01.06.2012) and as exemplified in the examples.

HMOs are generally composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of Sia. More than two hundred different HMOs have been identified so far. The most important ones are 2′-fucosyllactose (2′FL), lacto-N-neotetraose (LNnT), 3-fucosyllactose (3FL), difucosyllactose (DFL), Lacto-N-fucopentaose I (LNFP I), 3′Sialyllactose Sodium Salt (3′SL), 6′Sialyllactose Sodium Salt (6′SL), and Lacto-N-Tetraose (LNT).

HMOs can be isolated from breast milk, or they can be produced chemically or biochemically. HMOs are available commercially from a variety of producers.

For the purpose of the present invention the source of the HMO is not essential. It is clear that HMOs from different sources can be used.

Particularly suitable HMO's in all embodiments of the present invention are fucosylated HMO's such as in particular a1-2 respectively a1-3 fucosylated HMO's; sialylated HMO's such as in particular sialyllactoses, as well as the neutral core HMO's as well as any mixtures thereof.

Particularly preferred fucosylated HMO's according to the present invention are 2′-fucosyllactose (2′FL; CAS No: 41263-94-9), 3-fucosyllactose (3FL; CAS No: 41312-47-4), difucosyllactose (DFL; also known as Lactodifucotetraose; CAS No: 20768-11-0) and Lacto-N-fucopentaose I (LNFP-1; CAS No: 7578-25-8).

Particularly preferred sialylated HMO's according to the present invention are sialyllactoses as well as salts thereof (preferably the sodium salts) such as in particular 3′sialyllactose (3′SL), 6′sialyllactose (6′SL) as well as the respective sodium salts thereof (CAS No's: 35890-39-2 (3′sialyllactose); 128596-80-5 (3′sialyllactose sodium salt); 35890-39-2 (6′sialyllactose); 157574-76-0 (6′sialyllactose sodium salt)).

Particularly preferred neutral core HMO's are built up from galactose, N-acetylglucosamine and glucose monosaccharide units, preferably linked through β-(1-3) or β-(1-4) bonds with each other. Preferably, in all embodiments herein, the galactose is D-galactose and the glucose is D-glucose.

In all embodiments of the present invention, preferably, the neutral core human milk oligosaccharides are tri-, tetra-, hexa-, octa- or decasaccharides, with the tri-, tetra- and hexasaccharides being particularly preferred. Most preferred are the tetrasaccharides.

In all embodiments of the present invention, it is further advantageous that the oligosaccharides in the neutral core HMO's are linear oligosaccharides, i.e. not branched.

Exemplary oligosaccharide of the neutral core HMO's according to the present invention are depicted below

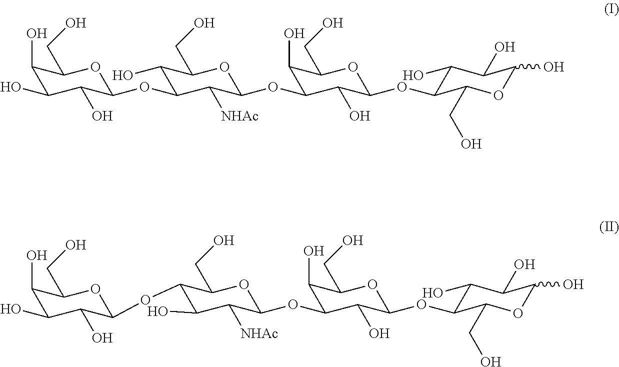 [SMILES: CC(=O)NC1C(OC2C(O)C(CO)OC(OC3C(CO)OC(O)C(O)C3O)C2O)OC(CO)C(O)C1OC1OC(CO)C(O)C(O)C1O ; CC(=O)NC1C(OC2C(O)C(CO)OC(OC3C(CO)OC(O)C(O)C3O)C2O)OC(CO)C(OC2OC(CO)C(O)C(O)C2O)C1O]

It is furthermore advantageous, that the oligosaccharides in the neutral core HMO's are characterized by a terminal D-galactose linked through a β-(1-3) or a β-(1-4) bond to a N-acetyl-d-glucosamine (GlcNAc) unit.

In addition, in all embodiments of the present invention, it is also preferred that the oligosaccharides in the neutral core HMO's are characterized by D-galactose linked through a β-(1-4) bond to a reducing end D-glucose.

Particularly suitable neutral core human oligosaccharides according to the present invention are listed in table 1

| No | Core name | Core structure |
|---|---|---|
| 1 | Lacto-N-triose II | GlcNAcβ1-3Galβ1-4Glc |
| 2 | lacto-N-tetraose (LNT) | Galβ1-3GlcNAcβ1-3Galβ1-4Glc |
| 3 | lacto-N-neotetraose (LNnT) | Galβ1-4GlcNAcβ1-3Galβ1-4Glc |
| 4 | lacto-N-hexaose (LNH) | Galβ1-3GlcNAcβ1-3(Galβ1-4GlcNAcβ1-6)Galβ1- |
| | | 4Glc |
| 5 | lacto-N-neohexaose (LNnH) | Galβ1-4GlcNAcβ1-3(Galβ1-4GlcNAcβ1-6)Galβ1- |
| | | 4Glc |
| 6 | para-lacto-N-hexaose | Galβ1-3GlcNAcβ1-3Galβ1-4GlcNAcβ1-3Galβ1- |
| | (para-LNH) | 4Glc |
| 7 | para-lacto-N-neohexaose | Galβ1-4GlcNAcβ1-3Galβ1-4GlcNAcβ1-3Galβ1- |
| | (para-LNnH) | 4Glc |
| 8 | lacto-N-octaose (LNO) | Galβ1-3GlcNAcβ1-3(Galβ1-4GlcNAcβ1-3Galβ1- |
| | | 4GlcNAcβ1-6)Galβ1-4Glc |
| 9 | lacto-N-neooctaose (LNnO) | Galβ1-4GlcNAcβ1-3(Galβ1-3GlcNAcβ1-3Galβ1- |
| | | 4GlcNAcβ1-6)Galβ1-4Glc |
| 10 | iso-lacto-N-octaose (iso-LNO) | Galβ1-3GlcNAcβ1-3(Galβ1-3GlcNAcβ1-3Galβ1- |
| | | 4GlcNAcβ1-6)Galβ1-4Glc |
| 11 | para-lacto-N-octaose (para- | Galβ1-3GlcNAcβ1-3Galβ1-4GlcNAcβ1-3Galβ1- |
| | LNO) | 4GlcNAcβ1-3Galβ1-4Glc |
| 12 | lacto-N-neodecaose (LNnD) | Galβ1-3GlcNAcβ1-3[Galβ1-4GlcNAcβ1-3(Galβ1- |
| | | 4GlcNAcβ1-6)Galβ1-4GlcNAcβ1-6]Galβ1-4Glc |
| 13 | lacto-N-decaose (LND) | Galβ1-3GlcNAcβ1-3[Galβ1-3GlcNAcβ1-3(Galβ1- |
| | | 4GlcNAcβ1-6)Galβ1-4GlcNAcβ1-6]Galβ1-4Glc |

Advantageously, in all embodiments of the present invention lacto-N-triose II, LNT, LNnT, LNH, LNnH, pLNH and pLNnH as well as mixtures thereof are used.

Most preferred neutral core HMO's in all embodiments of the present invention are Lacto-N-(neo)tetraoses, i.e. lacto-N-tetraose (CAS No: 14116-68-8) and/or lacto-N-neotetraose (CAS No: 13007-32-4).

The total amount of the HMO(s) according to the present invention in the compositions according to the present invention is preferably at least 0.01 wt.-%, more preferably at least 0.1 wt.-%, even more preferably at least 0.2 wt.-%, such as at least 0.3 wt.-% or at least 0.5 wt.-%, based on the total weight of the composition. In particular the total amount of the at least one HMO(s) according to the present invention in the compositions according to the present invention is preferably selected in the range from 0.01 to 10 wt.-%, more preferably in the range from 0.1 to 7.5 wt.-%, most preferably in the range from 0.2 to 5 wt.-%, based on the total weight of the composition. Further suitable ranges are from 0.25 to 5 wt.-%, from 0.5 to 4 wt.-%, from 0.1 to 3 wt.-%, from 0.25 to 3 wt.-% and from 0.3 to 3 wt.-%. Particularly preferred ranges according to the present invention are from 0.1 to 5 wt.-%, more preferably from 0.25 to 3 wt.-%, such as from 0.25 to 1.5 wt.-% 0.5 to 5 wt.-%, or 0.5 to 4 wt.-%

Preferably in all embodiments of the present invention the at least one HMO is selected from the group consisting of 2′-fucosyllactose (2′FL), 3-fucosyllactose (3FL), difucosyllactose (DFL), lacto-N-fucopentaose I (LNFP-I), 3′sialyllactose sodium salt (3′SL), 6′sialyllactose sodium salt (6′SL), lacto-N-neotetraose (LNnT), Lacto-N-tetraose (LNT), as well as mixtures thereof, more preferably from 3′Sialyllactose Sodium Salt (3′SL), 3-fucosyllactose (3FL), 6′sialyllactose sodium salt (6′SL), lacto-N-neotetraose (LNnT), Lacto-N-tetraose (LNT), difucosyllactose (DFL) as well as mixtures thereof, most preferably from 2′-fucosyllactose (2′FL) and Lacto-N-tetraose (LNT).

Preferably, in all embodiments of the present invention, the HMO(s) according to the present invention are present in an amount effective to increase the SPF (measured in vitro) of the composition by at least 20%, preferably by at least 30%, preferably by at least 50%, preferably by at least 100%, including all ranges and subranges therebetween such as, for example, 25% to 35%, 20% to 300%, 30% to 250%, 30% to 200%, and all ranges and subranges therebetween. It is well understood, that the difference is based on the respective composition not comprising the respective HMO(s).

It is well understood, that the compositions according to the present invention may comprise one or more HMO's. Preferably, however, the compositions according to the present invention comprise one or two, more preferably solely one HMO, such as most preferably selected from the group of 2′FL, 3FL, 3′SL, 6′SL, LNT and/or LNnT. The most preferred HMO in all embodiments of the present invention is LNT as it leads to a particular pronounced increase in the SPF.

The term ‘sunscreen active agent’ as used herein refers to compounds absorbing light in the UV-B and/or the UVA-range (also referred to UVA-, UVB- and broadband (UVA&B) filters). Preferably, such compounds have an E1/1 value (i.e. UV absorbance at a concentration of 1% at 1 cm thickness of absorbing layer at λₘₐₓ) of at least 150, more preferably of at least 180, most preferably of at least 190.

Preferred UVA filters generally absorb radiation in the 320 to 400 nm region of the ultraviolet spectrum. Examples of preferred UVA absorbers include dibenzoyl methanes or dialkylamino hydroxybenzoyl alkyl benzoate Preferred UVB filters absorb radiation in the 280 to 320 nm region of the ultraviolet spectrum. Examples of preferred UVB absorbers include cinnamates, diphenylacrylates, benzalmalonates, triazines and salicylates.

Preferred broadband UV filters provide substantial broad-spectrum protection from both UVA and UVB rays. Examples of preferred broadband absorbers include mincronized organic UV-filters such as methylene bis-benzotriazolyl tetramethylbutylphenol.

The sunscreen active agent(s) present in the compositions of the present invention can be organic sunscreen active agents (organic UV-filters) and/or inorganic sunscreen active agents (inorganic UV-filters) such as for example, physical blockers such as titanium dioxide or zinc oxide. Further, the sunscreen active agent(s) present in the compositions of the present invention can be soluble in water, soluble in non-aqueous material, and/or insoluble. Preferably, the sunscreen active agents according to the present invention are organic sunscreen active agents.

Preferably, in all embodiments of the present invention combinations of two or more, preferably of three or more, even more preferably of four and more different UV filters are used, such as combinations from 2 to 10, from 3 to 9 or from 4 to 7 different UV-filters. Advantageously, the two or more, preferably the three or more, even more preferably the four and more different UV-filters are selected from the group of organic UV-filters with all the preferences and definitions as given herein.

Preferably, in all embodiments of the present invention the total amount of all sunscreen active agent(s) in the compositions according to the invention is selected in the range from about 1 to about 50 wt.-% with respect to the total weight of the composition, preferably from about 3 to about 40 wt.-% with respect to the total weight of the composition, more preferably from about 5 to about 30 wt.-%, from about 10 to 25 wt.-% or from about 15 to 25 wt.-%, with respect to the total weight of the composition, including all ranges and subranges therebetween.

It is also preferred in all embodiments of the present invention, that the amount of all UV filters present in the composition according to the present invention is selected such that the SPF of the final composition (in vitro SPF measured as outlined in ISO 24443 Determination of sunscreen UVA photoprotection in vitro, 01.06.2012, as exemplified in the examples) is at least 10, preferably at least 15, more preferably at least 20.

Organic UV-filters particular useful herein include anthranilates; cinnamic derivatives; dibenzoylmethane derivatives; salicylic derivatives; camphor derivatives; triazine derivatives, such as those disclosed in U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzotriazole derivatives; benzalmalonate derivatives; benzimidazole derivatives; imidazolines; bisbenzoazolyl derivatives as disclosed in Patents EP 669 323 and U.S. Pat. No. 2,463,264; p-aminobenzoic acid (PABA) derivatives; methylenebis(hydroxyphenylbenzotriazole) derivatives as disclosed in U.S. Pat. Nos. 5,237,071 and 5,166,355, GB 2 303 549, DE 197 26 184 and EP 893 1 19; screening polymers and screening silicones, such as those disclosed in particular in Application WO 93/04665; dimers derived from α-alkylstyrene, such as those disclosed in Patent Application DE 198 55 649; 4,4-diarylbutadienes as disclosed in Applications EP 0 967 200, DE 197 46 654, DE 197 55 649, EP-A-1 008 586, EP 1 133 980 and EP 133 981; and their mixtures.

By way of illustration, mention may be made, as sunscreen active agents which are generally active in the UV-A and/or UV-B regions, denoted below under their INCI names, of: p-aminobenzoic acid (PABA) derivatives, in particular PABA, ethyl PABA, ethyl dihydroxypropyl PABA, ethylhexyl dimethyl PABA, glyceryl PABA or PEG-25 PABA, salicylic derivatives, in particular homosalate (HMS), ethylhexyl salicylate (EHS), dipropylene glycol salicylate, or TEA salicylate; dibenzoylmethane derivatives, in particular butyl methoxydibenzoylmethane (BMDBM), or isopropyl dibenzoylmethane; cinnamic derivatives, in particular ethylhexyl methoxycinnamate (OMS), isopropyl methoxycinnamate, isoamyl methoxycinnamate, cinoxate, DEA methoxycinnamate, diisopropyl methyl cinnamate, or glyceryl ethylhexanoate dimethoxycinnamate, β,β-diphenylacrylate derivatives, in particular octocrylene (OC) or etocrylene, benzophenone, in particular benzophenone-1, benzophenone-2, benzophenone-3 or oxybenzone, benzophenone-4, benzophenone-5, benzophenone-6, benzophenone-8, benzophenone-9, benzophenone-12, or n-hexyl 2-(4-diethylamino-2-hydroxybenzoyl)benzoate (DHHB), benzylidene camphor derivatives, in particular 3-benzylidene camphor, 4-methylbenzylidene camphor, benzylidene camphor sulphonic acid, camphor benzalkonium methosulphate, terephthalylidene dicamphor sulphonic acid, or polyacrylamidomethyl benzylidene, benzimidazole derivatives, in particular phenylbenzimidazole sulphonic acid (HS), or disodium phenyl dibenzimidazole tetrasulphonate, triazine derivatives, in particular bis-ethylhexyloxyphenol methoxyphenyl triazine (BEMT), ethylhexyl triazone (EHT), diethylhexyl butamido triazone (DBT), or 2,4,6-tris(diisobutyl 4′-amino-benzalmalonate)-s-triazine, benzotriazole derivatives, in particular drometrizole trisiloxane or insoluble organic sunscreen active agents such as methylene bisbenzotriazolyl tetramethylbutylphenol (MBBT) and tris-biphenyl triazine (A2B), which are generally sold in the form of aqueous dispersions of the active UV filter e.g. under the tradename PARSOL® MAX (MBBT) from DSM Nutritional Products or Tinosorb® A2B (A2B) from BASF, anthranilic derivatives in particular menthyl anthranilate, imidazoline derivatives, in particular ethylhexyl dimethoxybenzylidene dioxoimidazoline propionate, benzalmalonate derivatives, in particular polyorganosiloxane comprising benzalmalonate functional groups (e.g. commercially available under the INCI name: polysilicone-15), 4,4-diarylbutadiene derivatives, in particular 1,1′-dicarboxy (2,2′-dimethylpropyl)-4,4-diphenylbutadiene, and their mixtures.

Preferred organic sunscreen active agents (organic UV-filters) according to the present invention include homosalate (e.g. sold under the trade name “PARSOL® HMS”), ethylhexyl salicylate (e.g. sold under the trade name “PARSOL® EHS”), polysilicone-15 (sold in particular under the trade name “PARSOL® SLX”), ethylhexyl methoxycinnamate (e.g. sold under the trade name “PARSOL® MCX”), octocrylene (e.g. sold under the trade name “PARSOL® 340”), phenylbenzimidazole sulphonic acid (e.g. sold under the trade name “PARSOL® HS”) or a salt thereof, e.g. being formed during the preparation of the composition with a neutralization agent such as triethanolamine of sodium hydroxide, bis-ethylhexyloxyphenol methoxyphenyl triazine (e.g. sold under the trade name “PARSOL® SHIELD”), ethylhexyl triazone (e.g. sold under the trade name “PARSOL® EHT”), diethylhexyl butamido triazone (e.g. commercially available under the trade name Uvasaorb HEB), methylene bisbenzotriazolyl tetramethylbutylphenol (e.g. sold under the trade name “PARSOL® MAX”), tris-biphenyl triazine (e.g. sold under the trade name Uvinul A2B), butyl methoxydibenzoylmethane (sold in particular under the trade name “Parsol 1789”), diethylamino hydroxybenzoyl hexyl benzoate (e.g. sold under the trade name Uvinul A Plus), and their mixtures.

Most preferred organic sunscreen active agents (organic UV-filters) to be used in the compositions according to the present invention are ethylhexyl salicylate, polysilicone-15, ethylhexyl methoxycinnamate, octocrylene, phenylbenzimidazole sulphonic acid or a salt thereof, bis-ethylhexyloxyphenol methoxyphenyl triazine, ethylhexyl triazone, diethylhexyl butamido triazone, methylene bisbenzotriazolyl tetramethylbutylphenol, butyl methoxydibenzoylmethane, diethylamino hydroxybenzoyl hexyl benzoate as well as any mixtures thereof.

In all embodiments of the present invention, the (total) amount of the organic UV-filters in the compositions according to the present invention is selected in the range from 0.5 to 30 wt.-%, more preferably in the range from 1 to 25 wt.-%, and most preferably in the range from 1 to 25 wt.-%, such as from 1 to 15 wt.-%, based on the total weight of the composition. Further suitable amounts are selected in the range from 5 to 30 wt.-%, 10 to 30 wt.-%, 15 to 30 wt.-%, from 5 to 25 wt.-%, 10 to 25 wt.-% or from 15 to 25 wt.-%, based on the total weight of the composition.

In all embodiments of the present invention, the amount of ethylhexyl salicylate in the compositions according to the present invention is advantageously selected in the range from 0.5 to 10 wt.-%, preferably in the range from 0.5 to 9 wt.-%, 0.5 to 8 wt.-%, 0.5 to 7 wt.-%, 0.5 to 6 wt.-%, 0.5 to 5 wt.-%, such as for instance in the range from 1 to 10 wt.-%, from 2 to 10 wt.-%, from 3 to 10 wt.-% or from 2 to 7.5 wt.-%, based on the total weight of the composition.

In all embodiments of the present invention, the amount of polysilicone-15 in the compositions according to the present invention is advantageously selected in the range from 0.4 to 10 wt.-%, preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, from 1 to 2.5 wt.-% or from 1.5 to 2.5 wt.-%, based on the total weight of the composition.

In all embodiments of the present invention, the amount of ethylhexyl methoxycinnamate in the compositions according to the present invention is advantageously selected in the range from 0.5 to 10 wt.-%, preferably in the range from 0.5 to 9 wt.-%, 0.5 to 8 wt.-%, 0.5 to 7 wt.-%, 0.5 to 6 wt.-%, 0.5 to 5 wt.-%, such as for instance in the range from 1 to 10 wt.-%, from 2 to 10 wt.-%, from 3 to 10 wt.-% or from 2 to 7.5 wt.-%, based on the total weight of the composition.

In all embodiments of the present invention, the amount of octocrylene in the compositions according to the present invention is advantageously selected in the range from 0.5 to 15 wt.-%, preferably in the range from 0.5 to 10 wt.-%, 0.5 to 8 wt.-%, 1 to 10 wt.-%, 2 to 10 wt.-%, 3 to 10 wt.-%, such as for instance in the range from 5 to 10 wt.-%, from 6 to 10 wt.-% or from 7 to 10 wt.-%, based on the total weight of the composition.

In all embodiments of the present invention, the amount of phenylbenzimidazole sulphonic acid or a salt thereof in the compositions according to the present invention is advantageously selected in the range from 0.5 to 5 wt.-%, preferably in the range from 0.5 to 4 wt.-%, 0.5 to 3 wt.-%, 1 to 2.5 wt.-%, 1 to 3 wt.-% or 1 to 4 wt.-%, based on the total weight of the composition.

In all embodiments of the present invention, the amount of bis-ethylhexyloxyphenol methoxyphenyl triazine in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, from 1 to 2.5 wt.-% or from 1.5 to 2.5 wt.-%, based on the total weight of the composition.

In all embodiments of the present invention, the amount of ethylhexyl triazone in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, from 1 to 2.5 wt.-% or from 1.5 to 2.5 wt.-%, based on the total weight of the composition.

In all embodiments of the present invention, the amount of diethylhexyl butamido triazone in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, or from 1.5 to 3 wt.-%, based on the total weight of the composition.

In all embodiments of the present invention, the amount of methylene bisbenzotriazolyl tetramethylbutylphenol (based on a dispersion of approx. 45-55 wt.-% of MBBT as commercially available e.g. under the tradename PARSOL MAX) in the compositions according to the present invention is advantageously selected in the range from 0.5 to 10 wt.-%, preferably in the range from 1 to 10 wt.-%, more preferably in the range from 2 to 10 wt.-%, most preferably in the range from 2 to 6 wt.-% or from 3 to 6 wt.-%, based on the total weight of the composition.

In all embodiments of the present invention, the amount of butyl methoxydibenzoylmethane in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 2 to 5 wt.-%, from 3 to 5 wt.-% or from 3.5 to 5 wt.-%, based on the total weight of the composition.

In all embodiments of the present invention, the amount of diethylamino hydroxybenzoyl hexyl benzoate in the compositions according to the present invention is advantageously selected in the range from 0.5 to 10 wt.-%., preferably in the range from 0.5 to 9 wt.-%, 0.5 to 8 wt.-%, 0.5 to 7 wt.-%, 0.5 to 6 wt.-%, 0.5 to 5 wt.-%, 0.5 to 4 wt.-%, 0.5 to 3 wt.-%, 1 to 10 wt.-%, 1 to 9 wt.-%, 1 to 8 wt.-%, 1 to 7 wt.-%, 1 to 6 wt.-%, 1 to 5 wt.-%, 2 to 5 wt.-%, 3 to 5 wt.-%, such as for instance in the range from 2.5 to 6 wt.-%, from 3 to 6 wt.-%, from 4 to 6 wt.-% or from 3.5 to 5.5 wt.-%, based on the total weight of the composition.

Particularly preferred organic UV-filter combinations in all embodiments of the present invention encompass the combination of DHHB, EHT, BEMT, OMC, polysilicone-15, DBT and MBBT or BMDBM, EHS, OC and BEMT, optionally further in combination with HS as exemplified.

In one preferred embodiment, the compositions according to the present invention only comprise organic UV-filters.

In another preferred embodiment, the compositions further comprise one or more inorganic UV-filter.

The term ‘inorganic UV-filter’ as used herein refers to any metal oxide particles having UV-filter properties as defined above and are thus useful for incorporation into compositions as UV filters. Such inorganic UV-filters are well known to a person skilled in the art and are often referred to as micronized or microfine UV filters such as in particular micronized or microfine titanium dioxide and zinc oxide.

The particle size of such inorganic UV-filter is not particularly limited. In general, suitable (primary) particle sizes for an efficient UV-light absorption are selected in the range of 2 to 200 nm.

In all embodiments of the present invention, the (total) amount of the inorganic UV-filters in the compositions according to the present invention is preferably selected in the range from 0.5 to 30 wt.-%, more preferably in the range from 1 to 30 wt.-%, and most preferably in the range from 2 to 30 wt.-%, based on the total weight of the composition. Further suitable amounts are selected in the range from 1 to 25 wt.-%, 1 to 20 wt.-%, 1 to 15 wt.-%, 3.0 to 25 wt.-%, 4 to 25 wt.-%, 5 to 20 wt.-% or 10 to 25 wt.-%.

Even more advantageously, the (total) amount of inorganic titanium dioxide UV-filters in the compositions according to the present invention is selected in the range of 0.5 to 15 wt.-%, preferably in the range of 1 to 15 wt.-%, more preferably in the range of 2 to 10 wt.-%, most preferably in the range of 2 to 6 wt.-% or 3 to 6 wt.-%, based on the total weight of the composition.

Even more advantageously, the (total) amount of inorganic zinc oxide UV-filters in the compositions according to the present invention is selected in the range of 0.5 to 25 wt. %, preferably in the range of 1 to 20 wt.-%, more preferably in the range of 2 to 20 wt.-%, most preferably in the range of 5 to 20 wt.-% or 7.5 to 20 wt.-%, based on the total weight of the composition.

Preferably, if present in the compositions according to the present invention the ratio (w/w) of the titanium dioxide UV-filter(s) to the zinc oxide UV-filter(s) is selected in the range from 1:5 to 5:1, preferably from 1:2 to 2:1. Even more preferably the amount of zinc oxide in the compositions according to the present invention is higher than the amount of titanium dioxide such as in the range of 5:1 to 1.25:1, most preferably in the range of 2.5:1 to 1.25:1.

In one particular embodiment it is preferred that the compositions according to the present invention comprise solely inorganic UV filters.

According to the invention, it is advantageous that the inorganic UV-filters are surface-coated. The surface coating may comprise providing the metal oxide particles with a thin hydrophilic or hydrophobic inorganic or organic layer by methods known per se.

According to the present invention the different surface coatings can also comprise water. As a result of the surface treatment, the metal oxide is given a hydrophilic, amphiphilic or hydrophobic character.

Examples of inorganic surface coatings which are suitable for the purposes of the instant invention comprise aluminum oxide (Al₂O₃), aluminum hydroxide Al(OH)₃, aluminum oxide hydrate (also: Alumina, CAS-No.: 1333-84-2), sodium hexametaphosphate (NaPO₃)₆, sodium meta-phosphate (NaPO₃)ₙ, silicon dioxide (SiO₂) (also: Silica, CAS-No.: 7631-86-9), and iron oxide (Fe₂O₃). These inorganic surface coatings can be present on their own, in combination and/or in combination with organic coating materials, in particular as outlined below.

Examples of organic surface coatings which are suitable for use in the present invention include vegetable or animal aluminum stearate, fatty acids such as stearic acid and lauric acid, dimethylpolysiloxane (also: dimethicone), methylpolysiloxane (methicone), simethicone, trimethoxycaprylylsilane, triethoxycaprylylsilane, octyltrimethoxysilane and cetyl phosphates such as potassium cetyl phosphate as well as any mixtures thereof. These organic surface coatings can be present on their own, in combination and/or in combination with inorganic coating materials.

The crystalline form of the titanium dioxide may be of any crystal or amorphous type. For example, titanium dioxide may be any type of amorphous, rutil, anastase, brookite or a mixture thereof. Preferably, in all embodiments of the present invention, the crystalline form of the titanium dioxide is rutil.

It is furthermore preferred that the zinc oxide is a white powder consisting of zinc oxide present as wurtzite crystal structures.

Titanium dioxide UV-filters for use according to the present invention including any pre-dispersions thereof are e.g. available as PARSOL® TX (INCI: titanium dioxide, silica, dimethicone) at DSM Nutritional Products Ltd., Micro Titanium dioxide MT-01 at Tayca or TTO-551 at Ishihara Sangyo Kaisha. Also suitable is double coated titanium dioxide having an inner alumina coating and an outer simethicone coating e.g. commercially available as Eusolex T-2000 at EMD chemicals Inc./Rona.

Zinc oxide UV-filters for use according to the present invention including any pre-dispersions thereof are e.g. available from BASF as Z-Cote or Z-Cote HP1 (2% dimethicone coating), from Tayca as MZ-505S (5% methicone coating), or from DSM Nutritional Products Ltd as PARSOL® ZX (2-3.5% triethoxycaprylylsilane coating).

In a particular embodiment the compositions according to the present invention comprise as inorganic UV-filters at least one double coated titanium dioxide and at least one coated zinc oxide, most preferably in the absence of any further microfine metal oxides (i.e. one double coated titanium dioxide and one coated zinc oxide).

Most preferably in all embodiments according to the present invention the titanium dioxide UV-filter is either a double coated titanium dioxide, even more preferably a double coated titanium dioxide having an inner silica and an outer organic coating or a silica coated titanium dioxide.

Preferably, the inner silica coating layer of the double coated titanium dioxide consists of a minimum of 0.5 wt.-% of inorganic silica (based on titanium dioxide). More, preferably the inner coating layer consists of 0.5 wt.-% to 50 wt.-%, most preferably of 1 wt.-% to 20 wt.-% of inorganic silica (based on titanium dioxide).

The outer coating of the double coated titanium dioxide is preferably selected from the class of organic coatings such e.g. silicone oils (e.g. simethicones, methicones, dimethicones, polysilicone-15), alkyl silanes (e.g. octyl tri(m)ethoxy silane), olefinic acids (e.g. stearic acid), or polyols (e.g. glycerol) and can be applied to the titanium dioxide particle by methods known to a person skilled in the art e.g. described in FI57124. Preferably the outer coating is selected from the group consisting of simethicone, methicone, dimethicone, polysilicones-15, stearic acid and octyl trimethoxy silane. Most preferably the outer coating is dimethicone. Even more preferably, the amount of the outer coating layer is at least 0.25 wt.-% based on the titanium dioxide. Preferably the amount of the outer coating is selected in the range of 0.5 wt.-% to 50 wt.-%, most preferably in the range of 0.5 wt.-% to 10 wt.-%, based on the titanium dioxide.

In all embodiments of the present invention, the double coated titanium dioxide according to the present invention preferably has a titanium dioxide content selected in the range of 70-95 wt.-% and a silicon dioxide content selected in the range of 5-20 wt.-%, such as preferable a titanium dioxide content selected in the range of 80-90 wt.-% and a silicon dioxide content selected in the range of 10 to 15 wt.-%, with the proviso that the total content of titanium dioxide and silicone dioxide is selected in the range of 90-100 wt.-%.

Preferably, the double coated titanium dioxide has a mean primary particle size in the range from 2 to 100 nm, more preferably in the range of 5 to 50 nm, most preferably in the range of 10 to 25 nm and a secondary particle size between 0.025 and 1 μm, such as preferably between 0.05 and 0.075 μm.

Particularly suitable double coated titanium dioxide according to the present invention contains a rutil-type titanium dioxide (TiO₂) core with a double coating of silica (inner coating) and dimethicone (outer coating) and has titanium dioxide content of at least 75 wt.-%, preferably in the range from 82-87 wt.-% and a silicon dioxide content of at least 10 wt.-%, preferably in the range from 10.5 to 14.5 wt.-%, and a mean particle size distribution Dₙ50 of 25 to 100 nm, preferably 40 to 80 nm (analysed by Laser diffraction measurements with a Malvern Mastersizer 2000), which double coated titanium dioxide is e.g. commercially available as PARSOL® TX (INCI: titanium dioxide, silica, dimethicone) at DSM nutritional products Ltd.

Most preferably, in all embodiments according to the present invention, the zinc oxide is either uncoated or coated with triethoxycaprylylsilane. Most preferably, the zinc oxide is a white powder consisting of zinc oxide present as wurtzite crystal structures, coated with triethoxycaprylylsilane, which has a zinc oxide content of 96-98%, a triethoxycaprylylsilane content of 2-3.5% and a mean particle size of 90 to 130 nm (analysed by Laser diffraction measurements with a Malvern Mastersizer 2000) which is commercially available as PARSOL® ZX from DSM Nutritional Products Ltd.

If nothing else is stated in this specification any given parts and percentages are per weight and based on the total weight of the composition.

Preferably, the human milk oligosaccharide according to the present invention and the sunscreen active agent(s) are present in the compositions and methods of the invention in a weight ratio between 1:50 and 1:1, preferably between 1:40 and 1:7.5, and preferably between 1:30 and 1:5, such as 1:25 to 1:15. Preferably more sunscreen active agent(s) are present than HMO(s) on a weight basis.

As the compositions according to the invention are intended for topical application, it is well understood that they comprise a physiologically acceptable medium, i.e. a medium compatible with keratinous substances, such as the skin, mucous membranes, and keratinous fibres. In particular, the physiologically acceptable medium is a cosmetically acceptable carrier.

The term ‘cosmetically acceptable carrier’ as used herein refers to all carriers and/or excipients and/or diluents conventionally used in topical cosmetic compositions such as in particular in skin care preparations.

The exact amount of carrier will depend upon the actual level of the UV filters and any other optional ingredients that one of ordinary skill in the art would classify as distinct from the carrier (e.g., other active ingredients).

In an advantageous embodiment, the compositions according to the present invention comprise from 50% to 99%, preferably from 60% to 98%, more preferably from 70% to 98%, such as in particular from 80% to 95% of a carrier, based on the total weight of the composition.

In a particular advantageous embodiment, the carrier in all embodiments of the present invention consists furthermore of at least 30 wt. %, more preferably of at least 40 wt.-%, most preferably of at least 45 wt.-% of water, such as in particular of 50 to 90 wt.-%, from 50 to 80 wt.-%, from 50 to 79 wt.-% or from 50 to 60 wt.-% of water.

In particular, the compositions according to the present invention are cosmetic or pharmaceutical compositions, preferably cosmetic (non-therapeutic) compositions.

In one embodiment, the compositions according to the present invention are applied to mammalian keratinous tissue such as in particular to human skin or the human scalp and hair.

The term “cosmetic composition” as used in the present application refers to cosmetic compositions as defined under the heading “Kosmetika” in Rompp Lexikon Chemie, 10^{th} edition 1997, Georg Thieme Verlag Stuttgart, New York as well as to cosmetic compositions as disclosed in A. Domsch, “Cosmetic Compositions”, Verlag fur chemische Industrie (ed. H. Ziolkowsky), 4^{th} edition, 1992.

Preferred compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment, the compositions according to the invention are light-protective preparations (sun care products, sunscreens), such as sun protection milks, sun protection lotions, sun protection creams, sun protection oils, sun blocks or or day care creams with a SPF. Of particular interest are sun protection creams, sun protection lotions and sun protection milks.

The compositions of the invention (including the carrier) may comprise conventional adjuvants and additives, such as preservatives/antioxidants, fatty substances/oils, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing agents, as well as agents to improve elasticity and skin barrier and carriers and/or excipients or diluents conventionally used in compositions.

If nothing else is stated, the excipients, additives, diluents, etc. mentioned in the following are suitable for compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council, without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

The compositions in all embodiments of the present invention are emulsion compositions, i.e. compositions containing an oily phase and an aqueous phase such as in particular an O/W emulsions comprising at least one phosphate ester emulsifier. The amount of the oily phase (i.e. the phase containing all oils and fats) present in such emulsion compositions is preferably at least 10 wt.-%, such as in the range from 10 to 60 wt.-%, preferably in the range from 15 to 50 wt.-%, most preferably in the range from 15 to 40 wt.-%, based on the total weight of the composition.

According to one even more preferred embodiment, the compositions according to the present invention as outlined herein are O/W emulsions comprising an oily phase dispersed in an aqueous phase in the presence of at least a phosphate ester emulsifier. The preparation of such O/W emulsions is well known to a person skilled in the art and illustrated in the examples.

In one advantageous embodiment, the O/W emulsifier is a phosphate ester emulsifier. Among the preferred phosphate ester emulsifier are C8-10 alkyl ethyl phosphate, C9-15 alkyl phosphate, C20-22 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate. Particular preferred phosphate ester emulsifiers in all embodiments of the present invention are C8-C25 alkyl phosphates, such as in particular C20-22 alkyl phosphates and cetyl phosphates, and in particular the respective potassium salts thereof. A particular preferred phosphate ester emulsifier according to the invention is potassium cetyl phosphate e.g. commercially available as Amphisol® K at DSM Nutritional Products Ltd Kaiseraugst.

Further suitable O/W emulsifiers which could be used in combination with the phosphate ester emulsifier encompass PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self-emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C10-30 alkyl acrylate crosspolymer, acrylates/steareth-20 methacrylate copolymer, PEG-22/dodecyl glycol copolymer, PEG-45/dodecyl glycol copolymer, and mixtures thereof.

Another particular suitable class of O/W emulsifiers to be used in combination with the phosphate ester emulsifier are non-ionic self-emulsifying systems derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable to be used in combination with the phosphate ester emulsifier are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Lubrizol.

Also suitable to be used in combination with the phosphate ester emulsifier are polyalkylenglycolether such as Brij 72 (Polyoxyethylen(2)stearylether) or Brij 721 (Polyoxyethylene (21) Stearyl Ether e.g. available at Croda.

Particularly advantageous additional O/W emulsifiers to be used in combination with the phosphate ester emulsifier are one or more of Polyglyceryl-3 Methylglucose Distearate, Lauryl Glucoside (and), Glyceryl Sterate Citrate, Sodium Cetearyl Sulfate, Cetearyl Glucoside; Polyglyceryl-6 Stearate (and) Polyglyceryl-6 Behenate, Cetearyl Olivate (and) Sorbitan Olivate, Arachidyl Alcohol (and) Behenyl Alcohol (and) Arachidyl Glucosides, Cetearyl Alcohol (and) Coco-Glucoside, Coco-Glucoside (and) Coconut Alcohol, PEG-100 Stearate (and) Glyceryl Stearate, Sodium Stearoyl Glutamate, Steareth-20, Steareth-21, Steareth-25, Steareth-2, Ceteareth-25 and Ceteareth-6 (all listed by their INCI names).

In one embodiment it is particularly preferred in accordance with the invention if the composition comprises a cetyl phosphate, preferably potassium cetyl phosphate as sole emulsifier.

In another embodiment it is particularly preferred in accordance with the invention if the composition comprises a Polyglyceryl-6 Stearate; Polyglyceryl-6 Behenate, PEG-100 Stearate, and/or Glyceryl Stearate Citrate, preferably Polyglyceryl-6 Stearate and/or Polyglyceryl-6 Behenate as emulsifier together with a cetyl phosphate emulsifier, more preferably with potassium cetyl phosphate as a co-emulsifier.

The at least one phosphate ester emulsifier is preferably used in an amount of 0.1 to 10 wt. % such as in particular in the range from 0.25 to 7.5 wt.-% such as most in particular in the range from 0.5 to 4 wt.-% or in the range from 0.5 to 3 wt.-%, based on the total weight of the composition.

If the phosphate ester emulsifier with all the definitions and preferences as given herein is used as a co-emulsifier, the amount is preferably selected in the range of 0.1 to 1 wt. % such as in particular in the range of 0.25 to 0.75 wt.-% such as most in particular in the range of 0.3 to 0.7 wt.-% or in the range of 0.4 to 0.6 wt.-%, based on the total weight of the composition.

The concentration of additional O/W emulsifiers to be used in combination with the phosphate ester emulsifier according to the present invention is preferably selected in the range of 0.4 to 10 wt. % such as in particular in the range from 0.8 to 8.0 wt.-% such as most in particular in the range from 1.0 to 6 wt.-% or in the range from 1.5 to 5 wt.-%, based on the total weight of the composition.

The emulsion compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 7 wt.-%, such as most in particular in the range of 1 to 5 wt.-%, based on the total weight of the composition. Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), polyhydroxystearic acid, glyceryl stearate, glyceryl myristate (Estol 3650), hydrogenated coco-glycerides (Lipocire Na10) as well as mixtures thereof.

In all embodiments of the present invention, it is particular advantageous if the emulsion compositions according to the present invention also comprises cetyl alcohol, cetearyl alcohol, and/or glycerylstearate, most preferably cetearyl alcohol, and/or glycerylstearate.

Advantageous embodiments of the emulsion composition of the present invention also include those wherein the composition comprises one or more oils selected from butylene glycol dicaprylate/dicaprate, phenethyl benzoate, C₁₂-C₁₅ alkyl benzoate, dibutyl adipate, diisopropyl sebacate, dicaprylyl carbonate, di-C₁₂₋₁₃ alkyl tartrates, diethylhexyl syringylidene malonates, hydrogenated castor oil dimerates, triheptanoin, C₁₂₋₁₃ alkyl lactates, C₁₆₋₁₇ alkyl benzoates, propylheptyl caprylates, caprylic/capric triglycerides, diethylhexyl 2,6-naphthalates, octyldodecanol, ethylhexyl cocoates, and phenethyl alcohol. Preferably, the composition according to the present invention comprises as oil(s) dibutyl adipate, phenylethyl benzoate, butylene glycol, phenethyl benzoate, phenethyl alcohol, C₁₂-C₁₅ alkylbenzoate, diisopropyl sebacate, as well as mixtures thereof.

In a still further advantageous aspect of the invention, the compositions of the present invention further comprise a preservative and/or a preservative booster, preferably selected from the group consisting of ethanol, phenoxyethanol, sodium benzoate, potassium sorbate, phenethyl alcohol, hydroxyactophenone, ethylhexylglycerin, hexylglycerin, glyceryl caprylate, 1,3-propanediol, 1,2-pentanediol, 1,2-hexanediol, 1,2-octanediol (caprylyl glycol), 1,2-decanediol and 2-methyl-1,3-propanediol as well as mixtures thereof, most preferably selected from the group of phenoxyethanol and ethylhexyl glycerine as well as mixtures thereof. When present, the preservative respectively the preservative booster is preferably used in an amount of 0.01 to 5 wt. %, more preferably in an amount of 0.05 to 4 wt.-%, most preferably in an amount of 0.1 to 3.0 wt.-%, based on the total weight of the composition.

It is advantageous in accordance with the invention if the emulsion compositions according to the present invention further comprise one or more alkanediols from the group 1,3-propanediol, 1,2-pentanediol, 1,2-hexanediol, 1,2-octanediol, 1,2-decanediol and 2-methyl-1,3-propanediol. When present, the one or more alkanediols is preferably used in an amount of 0.1 to 7 wt. %, more preferably in an amount of 0.5 to 7 wt.-%, most preferably in an amount of 1.0 to 5.0 wt.-%, based on the total weight of the composition.

It is further advantageous in accordance with the invention if the emulsion composition of the invention comprises phenoxyethanol, ethylhexylglycerin, glyceryl caprylate and/or phenethyl alcohol, preferably phenoxyethanol and/or ethylhexylglycerin or glyceryl caprylate and/or phenethyl alcohol, most phenoxyethanol and ethylhexylglycerin or glyceryl caprylate and phenethyl alcohol

In another advantageous aspect, the emulsion compositions according to the present invention are free of any parabenes, benzethoniumchlorid, piroctone olamine, lauroylarginat, methylisothiazolinon, chlormethylisothiazolinon, bronopol, benzalkoniumchloride, formaldehyd releasing compounds, salicylic acid, triclosan, DMDM hydantoin, chlorphenesin and IPBC (lodopropinylbutyl carbamate).

The emulsion compositions according to the invention may further contain one or more emollients which soothe and soften the skin. As an example, the emollient may be silicone (dimethicone, cyclomethicone), vegetable oils (grape seed, sesame seed, jojoba, etc.), butters (cocoa butter, shea butter), and petrolatum derivatives (petroleum jelly, mineral oil).

In another aspect, the emulsion compositions of the invention may comprise one or more fragrances selected from limonene, citral, linalool, alpha-isomethylionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic diester, cinnamal, amyl salicylate, alpha-amylcinnamaldehyde, alpha-methylionone, butylphenylmethylpropional, cinnamal, amylcinnamyl alcohol, anise alcohol, benzoin, benzyl alcohol, benzyl benzoate, benzyl cinnamate, benzyl salicylate, bergamot oil, bitter orange oil, butylphenylmethylpropional, cardamom oil, cedrol, cinnamal, cinnamyl alcohol, citronellyl methylcrotonate, citrus oil, coumarin, diethyl succinate, ethyllinalool, eugenol, Evernia furfuracea extract, Evernia prunastri extract, farnesol, guaiacwood oil, hexylcinnamal, hexyl salicylate, hydroxycitronellal, lavender oil, lemon oil, linalyl acetate, mandarin oil, menthyl PCA, methyl hexadecan, nutmeg oil, rosemary oil, sweet orange oil, terpineol, tonkabean oil, triethyl citrate, vanillin.

The emulsion compositions of the invention may advantageously comprise moisturizers. Moisturizers are compounds or mixtures of compounds which give cosmetic compositions the quality, after application to or distribution on the skin surface, of reducing the loss of moisture of the stratum corneum (also called transepidermal water loss (TEWL)) and/or of positively influencing the hydration of the stratum corneum.

Non-limiting examples of advantageous moisturizers for use in the emulsion compositions of the present invention include glycerol, lactic acid and/or lactates, especially sodium lactate, butylene glycol, propylene glycol, 1,3-propanediol, biosaccharide gum-1, Glycine soya, ethylhexyloxyglycerol, pyrrolidonecarboxylic acid, saccharide isomerate and urea. Of further advantage in particular is the use of polymeric moisturizers from the group of the polysaccharides which are water-soluble and/or swellable in water and/or gellable with the aid of water. Especially advantageous, for example, are hyaluronic acid, chitosan and/or a fucose-rich polysaccharide which is registered in Chemical Abstracts under the registry number 178463-23-5 and is available, for example, under the Fucogel® 1000 name from the company SOLABIA S.A. Moisturizers may also be used advantageously as active antiwrinkle ingredients for protection from changes to the skin of the kind occurring in skin aging, for example.

Particularly advantageous moisturizers to be used in the emulsion compositions according to the present invention are glycerin, sodium hyaluronate, saccharide isomerate and/or 1,3-propanediol, more preferably in a total amount selected in the range of 0.1 to 5 wt.-%, more preferably in the range from to 0.1 to 3 wt.-%, even more preferably in the range of 0.5 to 3 wt.-%, based on the total weight of the composition.

The emulsion compositions of the invention may further comprise advantageously, although not mandatorily, fillers which have the effect, for example, of further improving the sensorial and cosmetic properties of the formulations and evoking or intensifying a velvety or silky skin sensation, for example. Advantageous fillers in the sense of the present invention are starch and starch derivatives (such as tapioca starch, distarch phosphate, aluminum or sodium starch octenylsuccinate, and the like, for example), Valvance pigments which have neither primarily UV filter effect nor coloring effect (such as Valvance Touch 210 or 250 for example) and/or Aerosils® (CAS No. 7631-86-9) and/or talc and/or polyethylene, nylon, and silica dimethyl silylate.

The water phase of the emulsion compositions of the invention may advantageously comprise customary cosmetic auxiliaries, such as, for example, alcohols, particularly those of low C number, preferably ethanol and/or isopropanol, or polyols of low C number, and also ethers thereof, preferably propylene glycol, glycerol, electrolytes, self-tanning agents, and also, in particular, one or more thickeners, which may be advantageously selected from the group of silicon dioxide, aluminum silicates, polysaccharides and/or derivatives thereof, e.g., hyaluronic acid, xanthan gum, hydroxypropylmethylcellulose, particularly advantageously from the group of polyacrylates, preferably a polyacrylate from the group referred to as Carbopols, examples being carbopols of types 980, 981, 1382, 2984, and 5984, in each case individually or in combination. Further thickeners advantageous in accordance with the invention are those having the INCI designation Acrylates/C10-30 Alkyl Acrylate Crosspolymer (e.g., Pemulen TR 1, Pemulen TR 2, Carbopol 1328 from Lubrizol) and also Aristoflex AVC (INCI: Ammonium Acryloyldimethyltaurate/VP Copolymer) as well as Simugel NS (INCI: Hydroxyethyl Acrylate/Sodium Acryloyldimethyl Taurate Copolymer & Squalane & Polysorbate 60).

It is further preferred in accordance with the invention if the composition comprises a thickener. Suitable thickeners to be used according to the present invention encompass xanthan gum, crosslinked acrylate/C10-C30 alkyl acrylate polymer, hydroxyethyl acrylate/sodium acryloyldimethyl taurate copolymer, and/or vinylpyrrolidone/hexadecane copolymer, preferably xanthan gum and/or hydroxyethyl acrylate/sodium acryloyldimethyl Taurate, most preferably xanthan gum.

The amount of xanthan gum, if present is preferably selected in the range of 0.1 to 1 wt.-%, more preferably in the range of 0.15 to 0.75 wt.-%, most preferably in the range of 0.2 to 0.4 wt.-%, based on the total weight of the composition.

The oil phase of the compositions of the invention may advantageously also comprise customary cosmetic auxiliaries, such as, for example, thickeners for the oil phase such as Dextrin Palmitate (Rheopearl KL2, Chiba Flour Milling), Dextrin Palmitate and C15-19 Alkane (TILAMAR Fits, DSM), Polyurethane-79 and Caprylic/Capric Triglyceride (Oilkemia 5S, Lubrizol), Ethylene/Propylene/Styrene Copolymer and Butylene/Ethylene/Styrene Copolymer and Pentaerythrityl Tetra-di-t-butyl Hydroxyhydrocinnamate and Squalane (Veggi Gel, Aiglon), Dibutyl Lauroyl Glutamide and Dibutyl Ethylhexanoyl Glutamide and Octyldodecanol (AJK-OD246, Ajinomoto).

The amount of the thickener (e.g. the active thickening polymer), if present is preferably selected in the range of 0.1 to 15 wt.-%, more preferably in the range of 0.5 to 10 wt.-%, most preferably in the range of 1.0 to 8.0 wt.-%, based on the total weight of the composition.

The emulsion compositions of the invention manage with a surprisingly small total amount of UV filters.

In another aspect, the emulsion composition may have an SPF of at least 15, preferably at least 20, most preferably of at least 30.

Advantageously in accordance with the invention, the emulsion composition of the invention comprises film formers. Film formers in the sense of the present invention are substances of various constitutions, and are characterized by the following properties: When a film former is dissolved in water or other suitable solvents, and when the solution is then applied to the skin, the film former, following evaporation of the solvent, forms a film which serves essentially to fix the UV-filters on the skin and thus to increase the water resistance of the product.

It is especially advantageous to select the film formers from the group of the polymers based on polyvinylpyrrolidone (PVP) Particular preference is given to copolymers of vinylpyrrolidone, as for example the PVP hexadecane copolymer and the PVP eicosene copolymer, which are available under the trade names Antaron V216 and Antaron V220 from Ashland.

Likewise advantageous are further polymeric film formers, such as, for example, sodium polystyrene sulfonate, which is available under the trade name Flexan 130 from National Starch and Chemical Corp., and/or polyisobutene, available from Rewo under the trade name Rewopal PIB1000. Examples of further suitable polymers are polyacrylamides (Sepigel 305), polyvinyl alcohols, PVP, PVP/VA copolymers, polyglycols, acrylate/octylacrylamide copolymer (Dermacryl 79) Likewise advantageous is the use of hydrogenated castor oil dimer dilinoleate (CAS 646054-62-8, INCI Hydrogenated Castor Oil Dimer Dilinoleate), which can be acquired from Kokyu Alcohol Kogyo under the name Risocast DA-H, or else PPG-3 benzyl ether myristate (CAS 403517-45-3), which can be acquired under trade name Crodamol STS from Croda Chemicals.

The emulsion compositions according to the invention in general have a pH in the range of 3 to 10, preferably a pH in the range of 4 to 8 and most preferably a pH in the range of 4 to 7.5. The pH can easily be adjusted as desired with suitable acids such as e.g. citric acid or bases such as NaOH or suitable amines such as tromethamine according to standard methods in the art.

The amount of the emulsion composition to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

The following examples are provided to further illustrate the compositions and effects of the present invention. These examples are illustrative only and are not intended to limit the scope of the invention in any way.

### EXAMPLES

### Short Description

The HMO's 2FL, 3′SL, LNT and LNnT were evaluated for their SPF boosting effect. For this purpose various sunscreens were prepared according to standard methods in the art as outlined in tables 1a, 2a and 3a.

### In Vitro SPF Measurement

The in vitro SPF test was performed on PMMA plates (HD6 plates from Helioscreen, 5 cm×5 cm, roughness of Sa=6 μm): 32.5 mg of the respective formulation (i.e. 1.3 mg/cm²) were applied homogenously onto the PMMA plates and dried for 15 minutes.

The in vitro SPF was determined using a Labsphere 2000 UV Transmittance Analyzer: each PMMA plate was measured 9 times at different points on the plate resulting in 27 data points. The result is calculated as the average of these 27 data points.

TABLE 1a
Formulation 1

| | | Conc. |
|---|---|---|
| Trade name | INCI name | [wt.-%] |
| Lexfilm Sun Natural MB | Capryloyl Glycerin/Sebacic Acid Copolymer | 1.5 |
| Lanette ® O | Cetearyl Alcohol | 1.0 |
| TEGIN ® M Pellets MB | Glyceryl Stearate | 0.4 |
| AMPHISOL ® K | Potassium Cetyl Phosphate | 0.5 |
| Cetiol ® B | Dibutyl Adipate | 9.5 |
| DL-alpha-Tocopheryl | Tocopheryl Acetate | 0.5 |
| Acetate | | |
| Uvinul A Plus | Diethylamino Hydroxybenzoyl Hexyl | 5.0 |
| | Benzoate | |
| PARSOL ® EHT | Ethylhexyl Triazone | 1.5 |
| PARSOL ® Shield | Bis Ethylhexyloxyphenol Methoxyphenyl | 2.0 |
| | Triazine | |
| PARSOL ® MCX | Ethylhexyl Methoxycinnamate | 4.0 |
| PARSOL SLX | Polysilicone-15 | 2.0 |
| Uvasaorb HEB | Diethylhexyl Butamido Triazone | 1.5 |
| X-Tend 226 | Phenethyl Benzoate | 5.5 |
| Dermosoft ® GMCY MB | Glyceryl Caprylate | 0.5 |
| TEGO CARE PBS 6 MB | Polyglyceryl-6 Stearate; Polyglyceryl-6 | 4.0 |
| | Behenate | |
| HMO | See table 1b | table 1b |
| HYA-ACT TM M | Sodium Hyaluronate | 0.1 |
| Phenethylalcohol Nat. | Phenethyl Alcohol | 1.0 |
| PARSOL ® MAX | Methylene Bis-Benzotriazolyl | 4.0 |
| | Tetramethylbutylphenol; Aqua; Decyl | |
| | Glucoside; Propylene Glycol; Xanthan Gum | |
| Water Dem | Aqua | Ad 100 |
| Rheocare ® XGN | XANTHAN GUM | 0.4 |
| TILAMAR ® PDO with | PROPANEDIOL | 2.0 |
| NØØVISTA ™ | | |

TABLE 1b
Results

| | | | SPF |
|---|---|---|---|
| HMO | Conc. HMO | SPF in vitro | boosting |
| Reference | — | 64.1 | — |
| Sialyllactose (3′SL) | 1% | 75.9 | +18% |
| Lacto-N-Neotetraose (LNnT) | 1% | 85.7 | +34% |

TABLE 2a
Formulation 2

| | | Conc. |
|---|---|---|
| Trade name | INCI name | [wt.-%] |
| AMPHISOL ® K | Potassium Cetyl Phosphate | 2.0 |
| Lanette O | Cetearyl Alcohol | 3.0 |
| PARSOL ® 1789 | Butyl Methoxydibenzoyl Methane | 4.0 |
| PARSOL ® EHS | Ethylhexyl Salicylate | 5.0 |
| PARSOL ® 340 | Octocrylene | 8.0 |
| PARSOL ® SHIELD | Bis Ethylhexyloxyphenol | 1.5 |
| | Methoxyphenyl Triazine | |
| Finsolv TN | C12-15 Alkyl Benzoate | 3.0 |
| Dub DIS | Diisopropyl Sebacate | 2.0 |
| Myritol 318 | Caprylic/Capric Triglyceride | 10.0 |
| WATER DEM. | Aqua | Ad 100 |
| Edeta BD | Disodium EDTA | 0.2 |
| Keltrol CG-T | Xanthan Gum | 0.2 |
| Glycerin 99.5% | Glycerin | 3.0 |
| Triethanolamine Care | Triethanolamine | 0.1 |
| HMO | See table 2b | table 2b |
| Euxyl PE 9010 | Phenoxyethanol; Ethylhexylglycerin | 1.0 |

TABLE 2b
Results

| | Conc. HMO | | |
|---|---|---|---|
| HMO | [wt.-%] | SPF in vitro | SPF boosting |
| Reference | — | 5.3 | — |
| Fucosyllactose (2′FL) | 1% | 10.0 | +89% |
| | 3% | 15.2 | +187% |
| Lacto-N-tetraose (LNT) | 1% | 15.6 | +194% |

TABLE 3a
Formulation 3

| | | Conc. |
|---|---|---|
| Trade name | INCI name | [wt.-%] |
| AMPHISOL ® K | Potassium Cetyl Phosphate | 2.0 |
| Lanette O | Cetearyl Alcohol | 3.0 |
| PARSOL ® 1789 | Butyl Methoxydibenzoyl Methane | 4.0 |
| PARSOL ® EHS | Ethylhexyl Salicylate | 5.0 |
| PARSOL ® 340 | Octocrylene | 8.0 |
| PARSOL ® | Bis Ethylhexyloxyphenol | 1.5 |
| SHIELD | Methoxyphenyl Triazine | |
| Finsolv TN | C12-15 Alkyl Benzoate | 3.0 |
| Dub DIS | Diisopropyl Sebacate | 2.0 |
| Myritol 318 | Caprylic/Capric Triglyceride | 5.0 |
| WATER DEM. | Aqua | Ad 100 |
| Edeta BD | Disodium EDTA | 0.2 |
| Keltrol CG-T | Xanthan Gum | 0.2 |
| Glycerin 99.5% | Glycerin | 3.0 |
| Triethanolamine | Triethanolamine | 1.3 |
| Care | | |
| PARSOL ® HS | Phenylbenzimidazole Sulfonic Acid | 2.0 |
| WATER DEM. | Aqua | 10.0 |
| HMO | see table 3b | table 3b |
| Euxyl PE 9010 | Phenoxyethanol; | 1.0 |
| | Ethylhexylglycerin | |

TABLE 3b
Results

| | Conc. HMO | | |
|---|---|---|---|
| HMO | [wt.-%] | SPF in vitro | SPF boosting |
| Reference | — | 11.4 | — |
| Fucosyllactose (2′FL) | 1% | 21.2 | +86% |
| | 3% | 23.3 | +104% |
| Lacto-N-tetraose (LNT) | 1% | 29.2 | +156% |

As shown in the tables 1 b, 2b and 3b the HMO's 2FL, 3SL, LNT and LNnT boost the in vitro SPF between 34% and 194%.

TABLE 4
Formulation 4

| Trade name | INCI Name | 1 (Control) | 2 |
|---|---|---|---|
| Sensanov WR | C20-22 Alkyl Phosphate | 2.0 | 2.0 |
| Lanette O | Cetearyl alcohol | 3.0 | 3.0 |
| PARSOL ® 1789 | Butylmethoxydibenzoyl | 4.0 | 4.0 |
| | methane | | |
| Parsol EHT | Ethylhexyl Triazone | 3.0 | 3.0 |
| PARSOL ® 340 | Octocrylene | 8.0 | 8.0 |
| HMS | Homosalate | 5.0 | 5.0 |
| Finsolv TN | C12-15 Alkyl benzoate | 3.0 | 3.0 |
| Dub DIS | Diisopropyl sebacate | 2.0 | 2.0 |
| Myritol 318 | Caprylic/Capric trigylceride | 10.0 | 10.0 |
| WATER DEM. | Aqua | 53.6 | 52.6 |
| Edeta BD | Disodium EDTA | 0.2 | 0.2 |
| Keltrol CG-T | Xanthan Gum | 0.2 | 0.2 |
| Glycerin 99.5% | Glycerin | 3.0 | 3.0 |
| 6′SL | Sialyllactose | | 1.0 |
| Euxyl PE 9010 | Phenoxyethanol, | 1.0 | 1.0 |
| | Ethylhexylglycerin | | |

| | | |
|---|---|---|
| SPF in vitro | 21.8 | 27.1 |
| SPF boosting | | +24.6% |

TABLE 5
Formulation 5 (no phosphate ester
emulsifier) - Comparative example

| | | 3 | |
|---|---|---|---|
| Tradename | INCI Name | (Control) | 4 |
| Tegocare 450 | Polyglyceryl-3 Methylglucose | 3.0 | 3.0 |
| | Distearate | | |
| Lanette O | Cetearyl alcohol | 3.0 | 3.0 |
| Uvinul A Plus | Diethylamino Hydroxybenzoyl | 5.0 | 5.0 |
| | Hexyl Benzoate | | |
| Parsol MCX | Ethylhexyl Methoxycinnamate | 8.0 | 8.0 |
| Parsol SLX | Polysilicone-15 | 3.0 | 3.0 |
| Uvasrob HEB | Diethylhexyl Butamido Triazone | 4.0 | 4.0 |
| Finsolv TN | C12-15 Alky benzoate | 3.0 | 3.0 |
| Dub DIS | Diisopropyl sebacate | 2.0 | 2.0 |
| Myritol 318 | Caprylic/capric triglyceride | 8.0 | 8.0 |
| WATER DEM. | Aqua | Ad 100 | Ad 100 |
| Edeta BD | Disodium EDTA | 0.2 | 0.2 |
| Keltrol CG-T | Xanthan Gum | 0.2 | 0.2 |
| Glycerin 99.5% | Glycerin | 3.0 | 3.0 |
| 3-Sialyllactose | Sialyllactose | | 1.0 |
| Euxyl PE 9010 | Phenoxyethanol, | 1.0 | 1.0 |
| | Ethylhexylglycerin | | |

| | | |
|---|---|---|
| SPF in vitro | 12.3 | 12.7 |
| SPF boosting | | +3.3% |

1. An emulsion composition with a phosphate ester emulsifier, wherein the emulsion composition comprises at least one sunscreen active agent and at least one human milk oligosaccharide.

2. The composition according to claim 1, wherein the at least one human milk oligosaccharide is present in an amount effective to increase SPF of the composition.

3. The composition according to claim 1, wherein the total amount of human milk oligosaccharides in the composition is selected in the range from 0.2 to 5 wt.-%, based on the total weight of the composition.

4. The composition according to claim 1, wherein the at least one human milk oligosaccharide is selected from the group consisting of 2′-fucosyllactose, 3-fucosyllactose, difucosyllactose, lacto-N-fucopentaose I, 3′sialyllactose sodium salt, 6′sialyllactose sodium salt, lacto-N-neotetraose and lacto-N-tetraose; or a mixture thereof.

5. The composition according to claim 1, wherein the amount of the phosphate ester emulsifier in the composition is selected in the range from 0.1 to 10 wt. %, based on the total weight of the composition.

6. The composition according to claim 1, wherein the phosphate ester emulsifier is a C8-C25 alkyl phosphate.

7. The composition according to claim 1, wherein the total amount of the at least one sunscreen active agent in the composition is selected in the range from 1 to 50 wt.-%, preferably from 3 to 40 wt. %, most preferably from 5 to 30 wt.-%, based on the total weight of the composition.

8. The composition according to claim 1, wherein the at least one sunscreen active agent is homosalate, ethylhexyl salicylate, polysilicone-15, ethylhexyl 2 methoxycinnamate, octocrylene, phenylbenzimidazole sulphonic acid or a salt thereof, bis-ethylhexyloxyphenol methoxyphenyl triazine, ethylhexyl triazone, diethylhexyl butamido triazone, methylene bisbenzotriazolyl tetramethylbutylphenol, tris-biphenyl triazine, butyl methoxydibenzoylmethane, diethylamino hydroxybenzoyl hexyl benzoate, or a mixture thereof.

9. The composition according to claim 1, wherein the composition comprises a carrier consisting of at least 30 wt. % of water.

10. The composition according to claim 1, wherein the composition is an O/W emulsion.

11. The composition according to claim 1, wherein the composition comprises the phosphate ester emulsifier as co-emulsifier together with one or more further emulsifiers.

12.-13. (canceled)

14. A method for increasing the sun protection properties (SPF) of an emulsion composition with a phosphate ester surfactant comprising at least one sunscreen active agent, said method comprising adding at least one human milk oligosaccharide to the composition in an amount effective to increase the SPF of the composition.

15. A method of preparing an emulsion composition with a phosphate ester emulsifier comprising adding at least one sunscreen active agent and at least one human milk oligosaccharide to the composition during preparation of the emulsion composition.

16. (canceled)

17. The composition according to claim 4, wherein the at least one human milk oligosaccharide is selected from the group consisting of 2′-fucosyllactose, difucosyllactose, 3′sialyllactose sodium salt, 6′sialyllactose sodium salt, lacto-N-neotetraose and lacto-N-tetraose; or a mixture thereof.

18. The composition according to claim 5, wherein the amount of the phosphate ester emulsifier in the composition is selected in the range from 0.5 to 4 wt.-%, based on the total weight of the composition.

19. The composition according to claim 6, wherein the C8-C25 alkyl phosphate is a C20-22 alkyl phosphate or a cetyl phosphate.

20. The composition according to claim 7, wherein the total amount of the at least one sunscreen active in the composition is selected in the range of 5 to 30 wt.-%, based on the total weight of the composition.

21. The composition according to claim 9, wherein the composition comprises a carrier consisting of at least 45 wt.-% of water.

22. The composition according to claim 11, wherein the one or more further emulsifiers comprises polyglyceryl-6 stearate, polyglyceryl-6 behenate, PEG-100 stearate, glyceryl stearate citrate, or a mixture thereof.
